(19) United States
(12) Patent Application Publication
(10) Pub. No.: US 2011/0230829 A1
(43) Pub. Date: Sep. 22, 2011

(54) Arterial Tamponade Device and Method

(76) Inventors: Patrick J. Fitzgerald, Poway, CA (US); R. Todd McKinney, Rancho Santa Fe, CA (US); John A. Simpson, Carlsbad, CA (US)

(21) Appl. No.: 12/751,731

(22) Filed: Mar. 31, 2010

Related U.S. Application Data
(60) Provisional application No. 61/315,761, filed on Mar. 19, 2010.
(60) Provisional application No. 61/315,766, filed on Mar. 19, 2010.

(51) Int. Cl. A61B 17/12

(52) U.S. Cl. 604/96.01

(57) ABSTRACT

An arterial tamponade device is expandable between a collapsed condition and a fully expanded position and has opposite ends which are spaced farther apart in the expanded position. A foot or pressure pad is associated with at least one end of the device. The device is inserted into a body cavity in its collapsed state and is released at a predetermined location so that opposite ends of the device are biased away from one another and engage and apply pressure to opposite wall areas of the body cavity before the device is fully expanded, with the pressure pad positioned to apply pressure to a predetermined tissue area which includes a blood vessel so as to occlude or partially occlude the vessel and reduce or cut off blood flow to the body cavity.

### RELATED APPLICATION

The present application claims the benefit of co-pending U.S. provisional patent application No. 61/315,761 filed on Mar. 19, 2010 and provisional patent application No. 61/315,766 filed on Mar. 19, 2010, the contents of each of which are incorporated herein by reference in their entirety.

### BACKGROUND

1. Field of the Invention

The present invention relates generally to tamponade devices used to block or restrict blood flow, and is particularly concerned with an intranasal tamponade device to reduce nasal bleeding by applying force to a blood vessel to partially or completely occlude blood flow.

2. Related Art

Bleeding during a surgical procedure is a problem for the surgeon because it limits visibility and can add to overall procedural time. This is particularly true in endonasal surgeries, both due to the fact that the nasal cavity is already narrow and dark, and due to the fact that the nasal cavity contains many small blood vessels that bleed easily, obscuring the surgical field. Currently, medication is used to reduce bleeding during surgery, or applied to the nose after surgery. Post-operative bleeding is also often controlled by nasal packing and packing devices such as gauze. Removal of such packing is typically quite painful and uncomfortable for the patient.

Because the nose has many small blood vessels fed from the sphenopalatine artery, cauterizing or severing of this artery is sometimes used for treating chronic nose bleeds.

There is therefore a need for a mechanism to improve visualization during surgery, particularly endonasal surgery, and to reduce bleeding more effectively without cauterization of arteries.

### SUMMARY

Embodiments described herein provide for a tamponade device to reduce bleeding during and after surgery or to reduce spontaneous nasal bleeding as a result of other nasal facial trauma.

According to one embodiment, a tamponade device is provided which is designed to be inserted into a body cavity in a compressed or collapsed state and which is configured to be expanded after insertion so that opposite ends of the device engage and apply pressure to opposite areas of the body cavity. At least one end of the device comprises a foot or pressure pad which is positioned to apply pressure to an area of the body cavity which includes an artery or other blood vessel, so as to occlude the vessel and reduce or cut off blood flow through the artery or blood vessel. This can be useful during surgery to reduce bleeding in the surgical field, after surgery to reduce post-operative bleeding, or to reduce bleeding as a result of trauma or various medical conditions.

The tamponade device in one embodiment comprises a relatively thin elongate member of resilient or shape memory material with at least one enlarged foot or pressure pad secured to one end. Both the rod and the foot are of suitable biocompatible materials appropriate for use in the body. The elongate member may be a wire, rod, or flat strip or ribbon of resilient plastic, metal or a balloon mechanism or the like. The enlarged foot or pad may be generally disc-shaped or oval, with an outwardly facing, tissue engaging surface, or may be of other shapes such as polygonal or irregular shapes, and the tissue engaging surface may be concave or convex. The foot may alternatively comprise a bent end portion of the elongate member. In one embodiment, an enlarged foot or pad is provided at both ends of the elongate member, and the opposite feet engage opposing regions of the body cavity. Alternatively, the opposite end of the elongate member may directly contact the opposing area of the body cavity to hold and support the device. The dimensions and material of the elongate member are such that the feet or pads can be deformed inwardly towards one another with the elongate member compressed into a U or V-like shape for insertion into a body cavity, for example using a medical grasping tool, a tubular sheath, an endoscope or a customized delivery system. When in the proper position, the tool or sheath can be withdrawn or the device can be pushed out of the sheath so that the elongate member springs out, forcing the feet apart into engagement with opposing regions of body tissue in the body cavity. The device is positioned so that the enlarged foot or pressure pad at the one end of the device engages a key point or area of the body that includes an artery or other blood vessel, applying pressure to occlude the blood vessel and reduce or cut off blood supply to the body region involved. The opposite end or foot engages an opposite area of the tissue so as to hold the device in place.

In one embodiment, the elongate member may be made from a shape memory metal or plastic material suitable for use in the body, such as nitinol (nickel and titanium alloy) or the like. The foot or feet may be of a suitable biocompatible material such as hydroxyapetite or a suitable biocompatible plastic or other material which may be injection molded around the end of the bent wire or ribbon.

The device can help to cut down bleeding during or after surgery, bleeding as a result of injury, or as result of certain medical conditions, by occluding an artery which provides blood supply to the area involved. In one embodiment the tamponade device is designed as an intranasal arterial tamponade device with the pressure pad on one leg positioned to occlude the sphenopalatine artery of the nose. The sphenopalatine artery (nasopalatine artery) passes through the sphenopalatine foramen into the cavity of the nose, at the back part of the superior meatus. The device can be positioned low in the nose out of the typical sinonasal surgical field, using a nasal endoscope device which may have position markings relative to the artery occluding foot to aid in proper positioning in the nasal cavity. The feet or pressure pads may be designed with a non-slip outer surface texture, for example with a roughened surface or a surface with plural dimples or bumps, to assist in holding the device in place, in addition to the spring pressure applied by the legs. Once placed in position, the non-slip texture of the feet, combined with the spring force applied by the flexible legs of the device, supports the feet in position while occluding or at least partially occluding the underlying artery.

In one embodiment, the feet can be drug-eluting so as to slowly release a drug over time, for example a drug which further reduces bleeding or which combats inflammation, infection and/or pain. For example, the feet in one embodiment have a coating of material which holds and elutes a drug into the tissue which they engage, similar to known drug-eluting stents.

According to another aspect, a method of temporarily applying pressure to an area of a body cavity wall including a blood vessel and occluding or substantially occluding the vessel to stop or reduce blood flow is provided, in which an expandable tamponade device is held in a compressed, unexpanded condition and inserted into a body cavity with a pressure pad at one end of the device oriented to face towards a predetermined region of the cavity wall which includes a blood vessel. The compressed tamponade device is released at a predetermined position in the body cavity so that it expands towards a fully expanded condition and opposite ends of the device engage and press against opposing wall regions of the body cavity before the device is fully expanded, with the pressure pad pressing against the predetermined region of the cavity wall including the blood vessel and applying sufficient pressure to at least partially occlude the blood vessel and reduce blood flow to the body cavity. The device may be held in the compressed condition by a suitable insertion tool or a nasal endoscope or insertion sheath until it reaches the desired position in the body cavity.

The embodiment of the device designed for intranasal use may also include a spring loaded turbinate extension from the elongate member which is secured at one end to the elongate member and is biased outwardly away from the elongate member in the relaxed condition of the device. The turbinate extension extends in a direction towards the footed end of the device, and is collapsed against the elongate member during insertion of the device. When the device is released from its compressed condition, the turbinate extension springs out away from the elongate member. In another embodiment, the turbinate extension may be of a malleable material that is physically bent into the desired position after insertion of the device. The turbinate extension is configured to retract or push the middle turbinate away from a surgical site when the enlarged foot is properly located so as to apply pressure to the sphenopalatine artery. This improves intraoperative visualization and can also be used to stent the middle meatus open in the postoperative period.

The arterial tamponade device described above is particularly useful as an intraoperative device to reduce bleeding in the operative field, particularly for endoscopic endonasal procedures which generally produce significant bleeding, so as to provide improved visualization of the surgical site. The device may also be used to reduce bleeding as a result of trauma or medical conditions such as nose bleed. One known treatment for nose bleeds, as an alternative to lengthy periods of nasal packing, is permanent vascular ligation or invasive arterial embolization which permanently blocks blood flow. Instead of permanent closing of an artery, the tamponade device could be installed to block blood flow temporarily, and then removed once bleeding is under control.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a front elevation view of a first embodiment of an arterial tamponade device in a relaxed, expanded condition;

FIG. 2 is a front elevation view of a modified tamponade device in a relaxed, expanded condition;

FIG. 3 is a front elevation view of the device of FIG. 1 or FIG. 2 in a partially compressed condition;

FIG. 4 is a side elevation view of the device in the compressed condition of FIG. 3;

FIG. 5 is a coronal cross-sectional view through a nasal cavity illustrating placement of the tamponade device of FIGS. 1 to 4 to occlude the sphenopalatine artery;

FIG. 6 is an enlarged view of a first foot portion of the device of FIG. 3 engaging the area of the lateral nasal wall including the sphenopalatine artery;

FIG. 7A is a side elevation view illustrating the device of FIGS. 1 to 6 confined in an introducer sheath or nasal endoscope for introduction and placement in the nasal cavity;

FIG. 7B is a view similar to FIG. 7A but illustrating the device partially pushed out of the sheath and expanding towards opposing areas of the nasal wall;

FIG. 7C is a view similar to FIGS. 7A and 7B but illustrating the device as it is pushed completely out of the sheath into an expanded condition pushing against opposing areas of the nasal wall;

FIG. 8 is a front elevation view of a modified intranasal arterial tamponade device in a partially compressed condition similar to FIG. 3;

FIG. 9 is a sagittal cross-sectional view through a nasal cavity illustrating placement of the modified tamponade device of FIG. 8 in the same position as illustrated in FIG. 5 for the embodiment of FIGS. 1 and 2;

FIG. 10 is a front elevation view of another modified tamponade device in a fully relaxed, expanded condition;

FIG. 11 is a front elevation view of intranasal arterial tamponade device of FIG. 10 in an introducer sheath prior to placement in a nasal cavity;

FIG. 12 is a coronal cross-sectional view through a nasal cavity illustrating placement of the tamponade device of FIGS. 10 and 11 to occlude the sphenopalatine artery and retract the middle turbinate out of a surgical field;

FIGS. 13A to 13M are illustrations of modified tamponade devices with different end foot designs;

FIG. 14A is a side elevation view illustrating another embodiment of an insertion device for placement of the tamponade device in a body cavity; and

FIG. 14B illustrates a modification of the insertion device of FIG. 14A.

### DETAILED DESCRIPTION

Certain embodiments as disclosed herein provide for a tamponade device configured for placement between opposing areas of a body cavity so as to apply pressure against a predetermined area of tissue, such as a wall of a cavity in the body, including a blood vessel, so as to temporarily occlude the blood vessel and stop or reduce blood flow through the vessel. In one embodiment, the device is designed for placement in a nasal cavity to occlude the sphenopalatine artery supplying blood to the nose.

After reading this description it will become apparent to one skilled in the art how to implement the invention in various alternative embodiments and alternative applications. However, although various embodiments of the present invention will be described herein, it is understood that these embodiments are presented by way of example only, and not limitation.

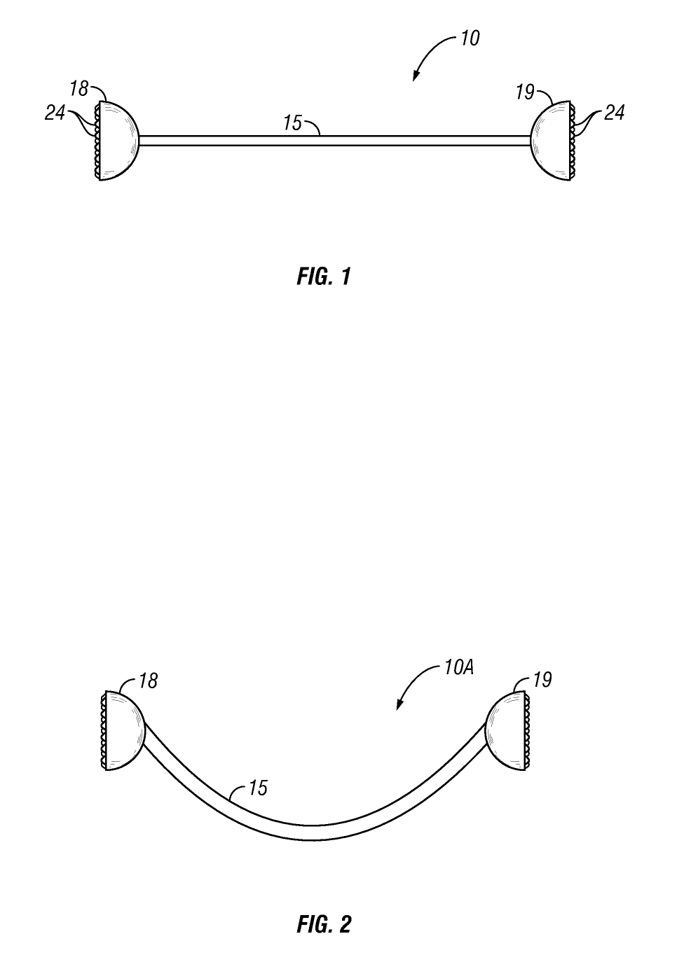

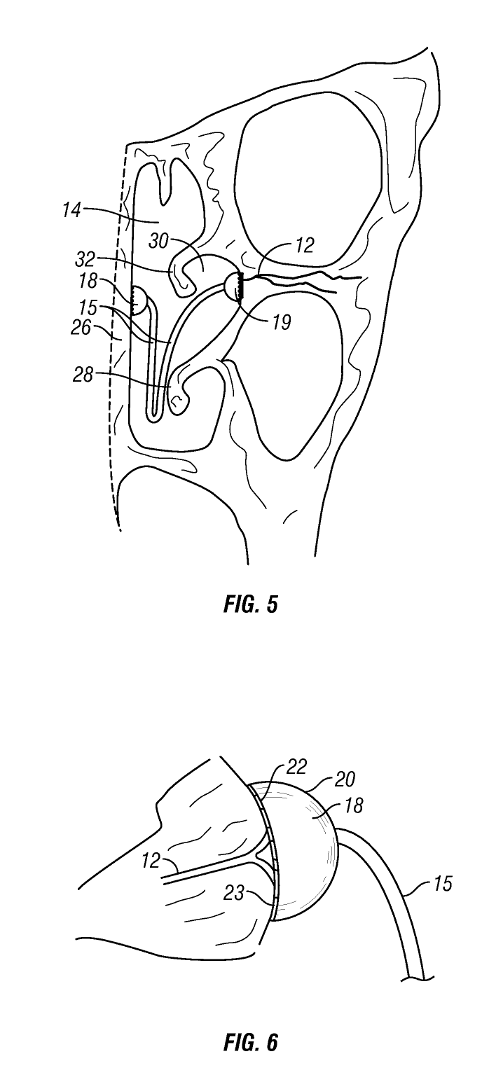

FIGS. 1, 3 and 4 illustrate a first embodiment of a tamponade device 10 which is designed to temporarily occlude a blood vessel such as an artery or vein, while FIGS. 5 and 6 illustrate the device in use. FIG. 2 illustrates a modified tamponade device 10A which is of slightly different configuration from device 10. In the illustrated embodiment, the device 10 or 10A is configured for placement in a nasal cavity 14 and is designed for temporarily occluding the sphenopalatine artery 12 of the nose. In alternative embodiments, the device may be of appropriate shape and dimensions for placement in other parts of the body or body cavities to apply pressure against blood vessels in tissue, such as cavity walls, so as to temporarily occlude the vessel or at least reduce blood flow through the vessel and reduce bleeding as a result of surgery, trauma or the like, as discussed in more detail below.

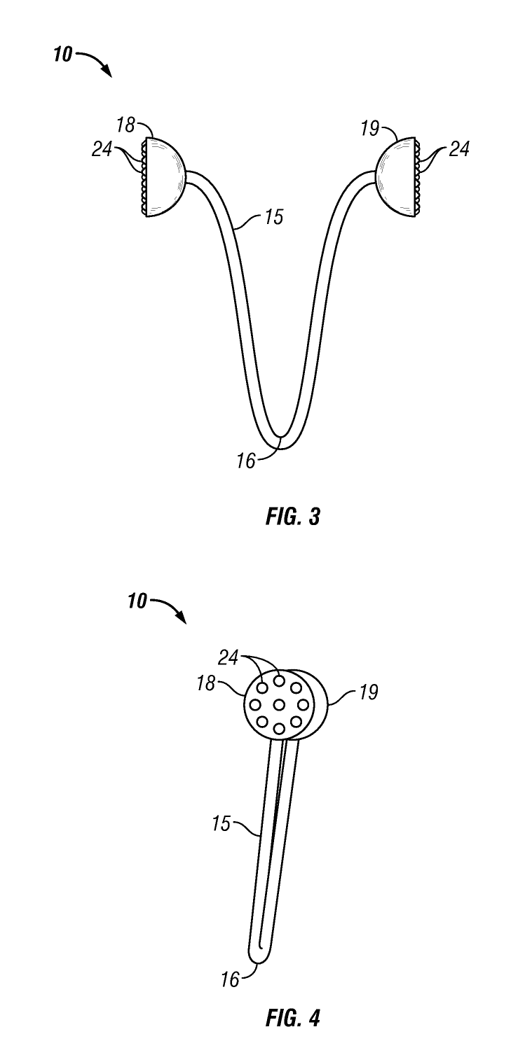

FIG. 1 illustrates device 10 in a relaxed, fully expanded condition, while FIGS. 3 and 4 illustrate the device in a partially compressed or distorted condition. Device 10 basically comprises a relatively thin elongate member 15 of resilient or shape-memory material with an enlarged foot or pressure applying pad 18, 19 secured to each end of member 15. Member 15 may be formed from a length of a suitable springy or resilient metal or plastic wire, rod, or flat ribbon or strip of a material which is biocompatible with body tissues, for example a shape memory alloy material such as nitinol or the like. In the embodiment of FIG. 1, the elongate member 15 is straight in the fully expanded, relaxed condition, while the modified embodiment of FIG. 2 is slightly curved when fully expanded. Other shapes may be used, such as a wide V-shape, U shape or the like. The elongate member may be solid or tubular.

In the illustrated embodiment, the feet or pads 18, 19 are substantially identical in shape and dimensions and each foot is of disc-like, round or oval shape with a rounded inner surface 20 and a cupped or concave outer surface 22 configured to engage an opposing surface of a body cavity, as illustrated in the enlarged sectional view of FIG. 6. In alternative embodiments, one foot may be larger than the other foot, and the feet may be of different shapes, as described in more detail below. The device may be compressed between the expanded, relaxed condition as illustrated in FIG. 1 and a compressed or deformed condition as illustrated in FIGS. 3, 4 and 7A, for example by pushing the feet or pads 18, 19 or opposite regions of member 15 towards one another, forming a generally V-like or looped shape with a bend 16 as in FIG. 3, for example.

The feet may be of any suitable biocompatible material such as hydroxyapatite or a biocompatible injection molded plastic or other material, and may be rigid or substantially rigid, and either solid or hollow. The shape of the outer, tissue engaging surface 22 is designed to substantially match the shape of the area of a body cavity which it is intended to engage, for close mating engagement and pressing against the area. Other shapes may be used for this surface, depending on the body cavity area to be engaged, such as convex, concave, substantially flat, or the like. In alternative embodiments, the material of feet 18, 19 may be slightly deformable to better match the shape of an opposing surface against which they are pressed on installation.

The body or tissue engaging surfaces 22 of the feet may also be designed as non-slip or slip-resistant surfaces. The surfaces may be roughened or may have a series of small protrusions 24 as indicated in FIGS. 1 to 4 so as to help grip and hold the device in place once positioned in the body cavity. In an alternative embodiment, the tissue engaging surfaces 22 may have indentions or dimples, or may have small openings or pores. The inner surfaces 20 are secured to the ends of the respective legs by adhesive or the like, or the feet may be injection molded over ends of the elongate member 15, which may be bent or hooked at its ends to better attach the feet. In one embodiment, the feet may be of metal and may be suitably welded to the ends of the legs. A twist or swivel joint (not illustrated) may be provided between the feet and legs to help in appropriate positioning of the outer, body engaging surfaces 22. The feet may also be an uninterrupted extension of the metal legs of the device.

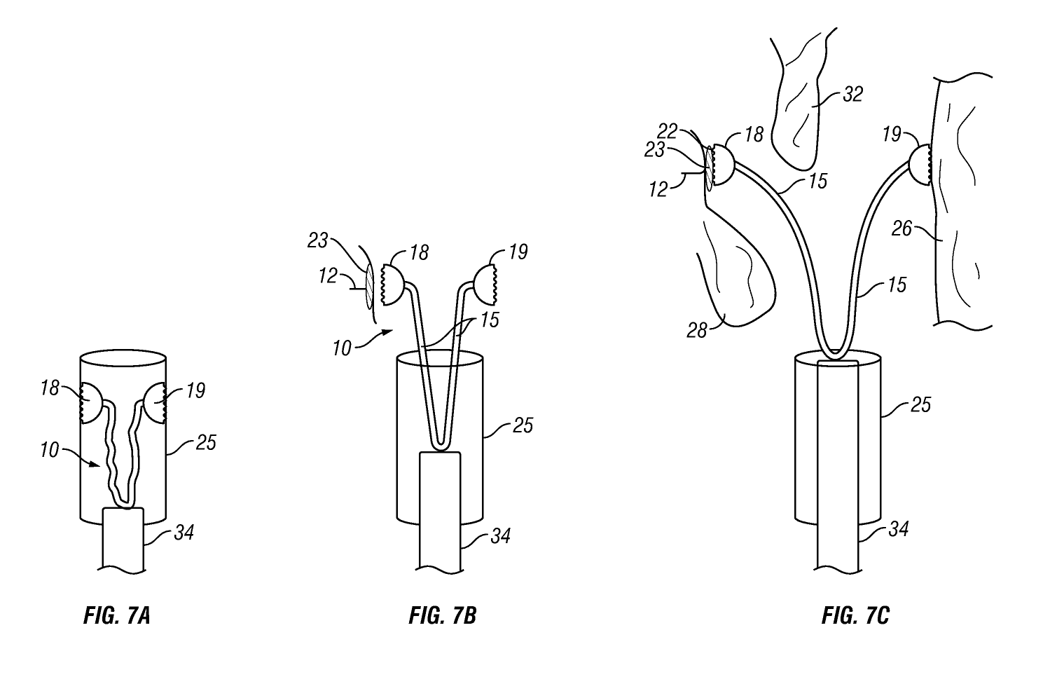

Device 10 may be positioned in a body cavity by means of a standard surgical grasping instrument, via an introducer such as a cylindrical sheath or endoscope 25, as illustrated in FIGS. 7A to 7C and described in more detail below, or via other customized delivery systems. Device 10 is designed to be collapsed from its original, unstressed or fully expanded shape as illustrated in FIG. 1 into a compact, compressed configuration as illustrated in FIG. 7A for installation purposes, so that it can be moved to a selected region in a body cavity without significantly contacting structures in the path to the desired region. The legs spring out towards the extended, unstressed position of FIG. 1 when released from the grasping instrument or introducer, and engage opposite wall regions of the body cavity while the device is still partially compressed, so that the partially compressed elongate member 15 applies a biasing force pressing the feet or pads 18, 19 against the opposite wall regions. In the embodiment illustrated in FIGS. 1 to 4, device 10 is designed for intranasal placement in a nasal cavity as illustrated in FIG. 5, with the spring force in the compressed elongate member or ribbon 15 urging the opposing feet 18, 19 against predetermined opposing areas of the nasal cavity. The device is positioned so that foot 18 is urged against predetermined area 23 of the lateral nasal wall including the sphenopalatine artery 12 and foot 19 bears against an opposite area of the nasal septum 26, with one of the legs extending over the inferior turbinate 28 and foot 18 positioned in the middle meatus area 30 between the inferior turbinate 28 and middle turbinate 32.

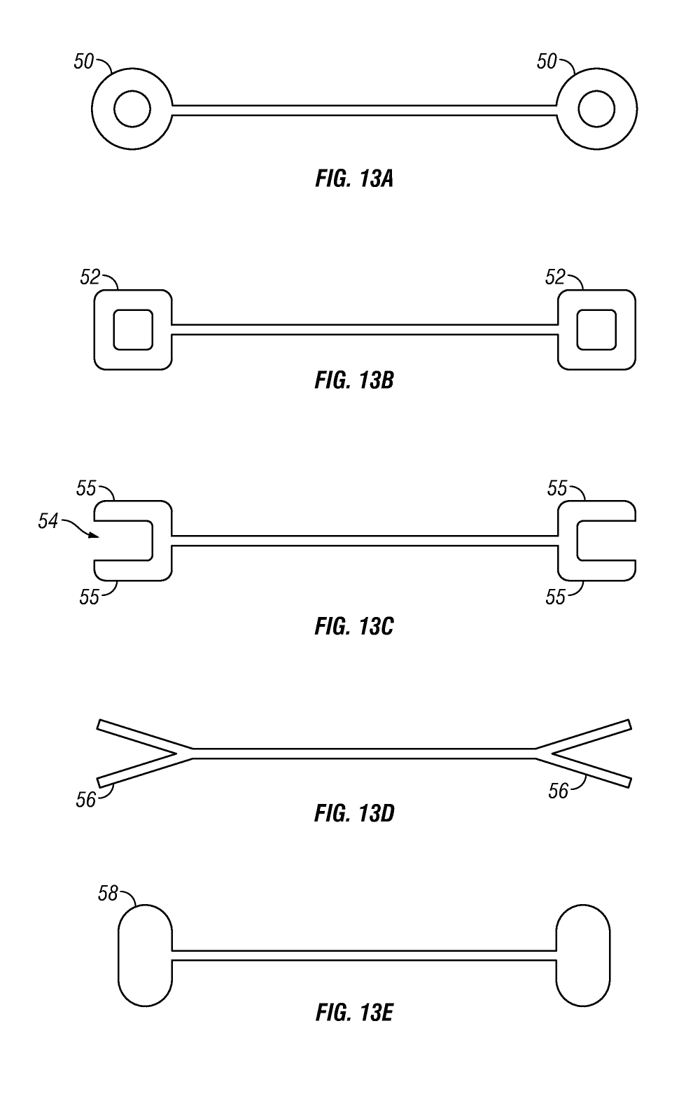

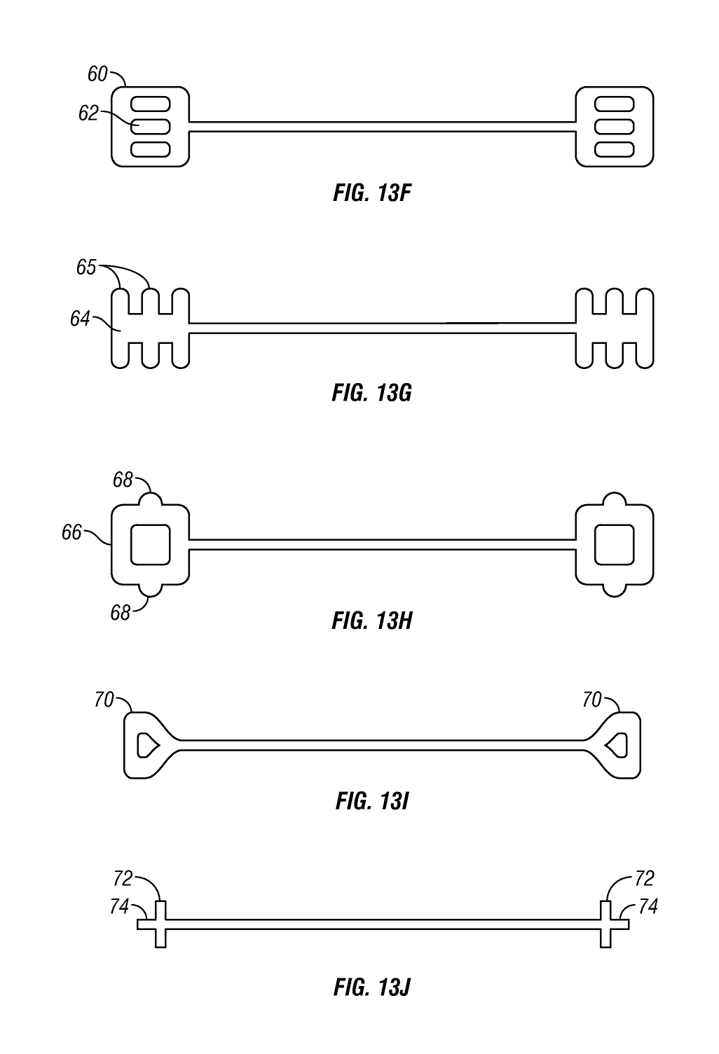

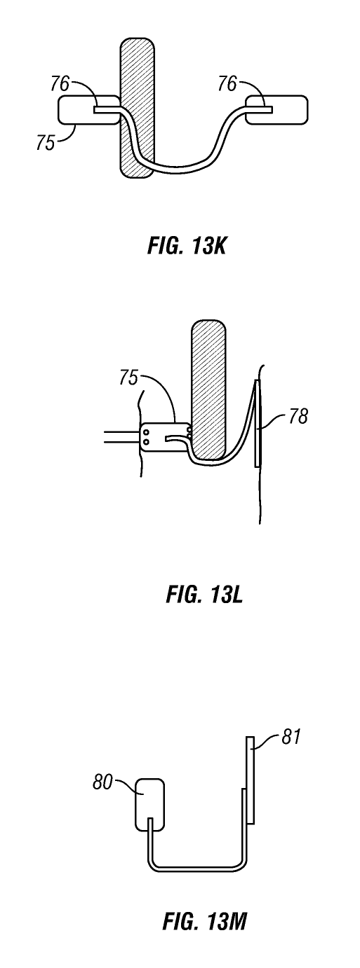

The foot 18 which is designed to engage the tissue surface including the sphenopalatine artery (or a blood vessel in a different body cavity in alternative embodiments) is suitably configured to apply sufficient pressure over an area of the artery to at least substantially occlude blood flow in the artery, and also to minimize trauma to the tissue. In the illustrated embodiment, this foot is circular or disc shaped with an arcuate tissue engaging surface. The tissue contacting surface may be convex or concave. The foot may be solid or hollow, and the tissue engaging surface may have an open center or core. Other peripheral shapes may be used for the feet, such as circular, oval, rectangular, polygonal, or irregular shapes, and the opposing inner and outer surfaces of the foot may be of the same or different shapes, and may be substantially flat, convex, concave, or the like. In other alternatives, the foot 18 may be a V-shaped or two pronged, fork-like wire extension at the end of the elongate member. The second foot is designed to assist in holding the device in position and may be of the same or different shape from foot 18. FIGS. 13A to 13M illustrate some alternative foot configurations and are described in more detail below.

The distance between the sphenopalatine artery and the septum in typical individuals is approximately 1.2-1.8 cm, with an average of 1.5 cm. The variability is on the septum side and to a lesser extent the skull size of the patient. The dimensions of device 10 in the fully expanded, relaxed condition of FIG. 1 are such that, when positioned and released at the desired location in nasal cavity 14 as in FIG. 3, the device cannot fully expand and the member 15 is still partially compressed or deformed inwardly from its original straight or partially curved shape so as to apply spring force against the opposing cavity surfaces via feet 18 and 19. The device may be of varying lengths. In one embodiment, the fully expanded spacing between the tissue engaging surfaces at the ends or feet of the device is of the order of 20 to 30 mm, and in one embodiment the fully expanded spacing is around 25 mm. The feet or pressure pads 18, 19 may be circular or oblong in shape and of the order of around 5 to 12 mm in cross-sectional dimension or diameter and 1 to 1.5 mm in thickness. In one embodiment, both pads have a diameter of around 7.5 mm. The artery side foot 18 and septum side foot 19 may be of different sizes in alternative embodiments. In one embodiment, the artery side foot was round with a diameter of around 7.5 mm while the septum side foot was oblong with a maximum dimension of around 10 mm. In one embodiment for use as a sphenopalatine artery tamponade, device 10 is designed so that foot 18 applies pressure of around 150 gm per sq. cm (around 2 psi) or more to the opposing tissue surface of the lateral nasal wall. In some cases, the device may be designed to apply a significantly higher amount of pressure. Devices of different sizes may be provided for different size nasal cavities or for use in different body cavities. The device is designed to be of relatively low profile when installed in the selected position in the nose.

When placed as illustrated in FIG. 5, the disc-like feet or pressure pads 18, 19 engage and apply pressure against the nasal mucosa and bone. The device is of relatively low profile when installed and is placed low in the nose out of the normal surgical field (see FIG. 9). Foot 18 is configured to exert pressure on the underlying sphenopalatine artery 12 so as to temporarily occlude the artery and cut off blood flow to the nasal cavity. This artery is a major blood supply to the nose and occluding it temporarily, for example during endoscopic sinonasal surgeries, reduces intra-operative bleeding and, more importantly, improve visualization and reduce surgical procedure times during such procedures. At the conclusion of surgery, the device 10 can be removed and discarded, or may be left in place for a time period after surgery if needed.

FIGS. 7A to 7C illustrate one embodiment of a delivery system for placing the arterial tamponade device 10 of FIGS. 1 to 4 in the nasal cavity at the desired location as illustrated in FIGS. 5 and 6. As illustrated in FIG. 7A, the device 10 is initially positioned within sheath 25 in a compressed, deformed state, with feet 18, 19 or opposite ends of the device pushed close together and the elongate member forming a loop or V-like shape, and the sheath is then inserted to the desired location within the nasal cavity. Markings on the introducer sheath may indicate the orientation to be used so as to correctly position one of the feet 18 to engage the key area 23 of the lateral nasal wall that includes the sphenopalatine artery 12. Once the sheath is at the desired position and at the correct orientation for positioning the feet, pusher 34 is used to push the device 10 out of the sheath and into position, as illustrated in FIGS. 7A and 7B. As the device exits the sheath, the opposite portions of the elongate member spring apart and the feet 18, 19 are pushed against opposing regions of the nasal cavity wall, specifically against the area 23 of the lateral nasal wall in the middle meatus which includes the sphenopalatine artery and against the opposing region of the nasal septum 26. The device is designed so that the pressure applied by the device frame or legs against area 23 is sufficient to hold the device in place and to occlude or at least substantially occlude blood flow from the sphenopalatine artery. The roughened consistency of the outer surfaces of the feet 18, 19 helps to hold the feet against slipping once they engage the walls.

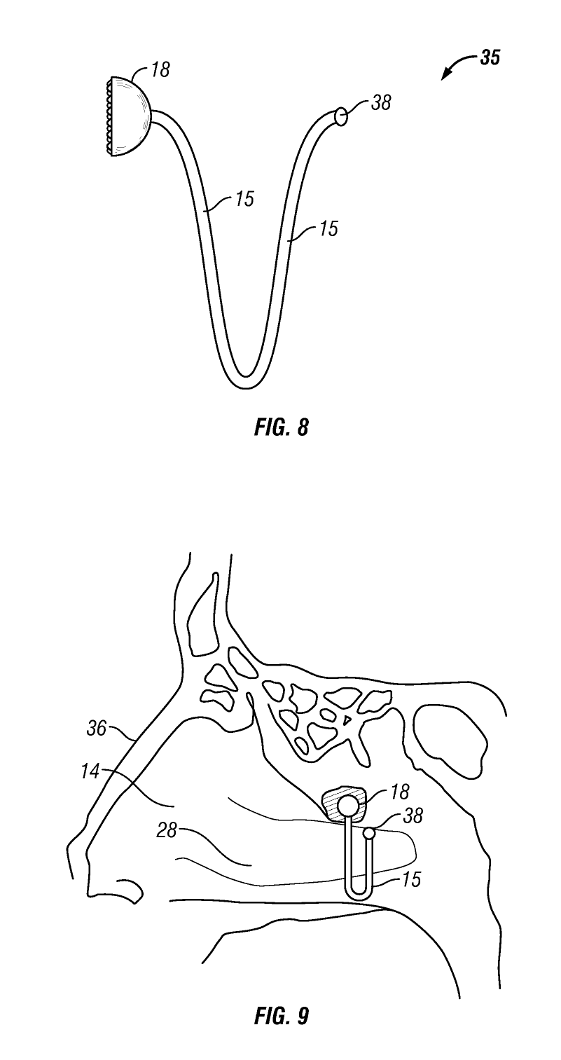

FIGS. 8 and 9 illustrate a modified tamponade device 35, with FIG. 8 illustrating the device in the same, partially compressed position as device 10 in FIG. 3, with the sectional view taken from a different direction. FIG. 9 is a side sectional view of the nose 36 and one nasal cavity 14. This shows how the device is positioned low in the nose and away from most sinonasal surgery sites. Some parts of the device 35 are identical to the previous embodiment, and like reference numbers are used for like parts as appropriate. The only difference is that one of the feet 19 of the previous embodiment is removed and replaced with a smaller foot 38 designed simply to contact the opposing structures of the nose to provide the desired fixation and support of the device. Alternatively, the foot 19 or 38 could be removed altogether and the frame leg 15 could directly contact the opposing nose structure or nasal septum, or could be bent into a loop or similar shape at the end for contacting the septum. In other alternative embodiments, foot 19 may be larger than foot 18 or the feet may be of different shapes.

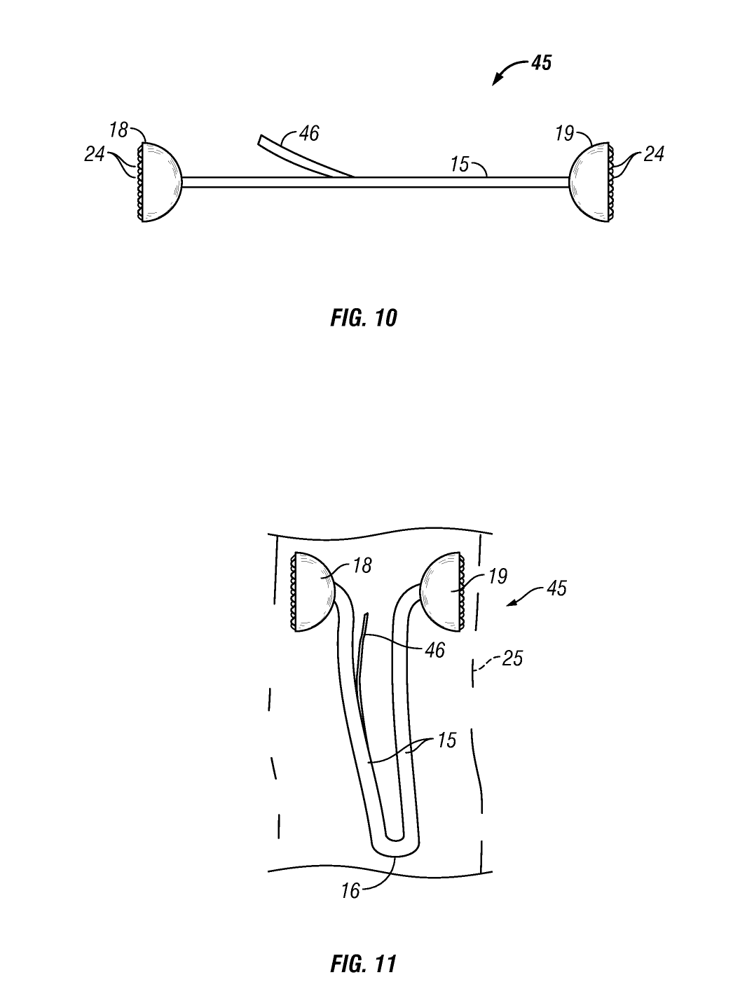

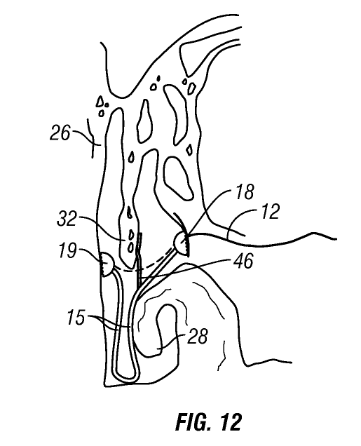

FIGS. 10 and 11 illustrate an arterial tamponade device 45 according to another embodiment, while FIG. 12 illustrates the device 45 positioned in the nasal cavity. This embodiment is similar to the embodiment of FIGS. 1 to 4, and like reference numbers are used for like parts as appropriate. However, device 45 has a turbinate extension 46 of the same thickness and material as elongate member 15. Extension 46 is secured to one face of the elongate member 15 and extends generally towards foot or pressure pad 18. Extension 46 is shaped so that it bends away from member 15 in the fully expanded, unstressed condition of FIG. 10. FIG. 11 illustrates the device in a collapsed, compressed state inside sheath 25, similar to the position for device 10 in FIG. 7A, in which opposite portions of member 15 are urged inwardly about bend 16 with turbinate extension 46 on the inner side of the resultant V or U shape. When the device 45 is released from the sheath 25 at the desired location in the nasal cavity, the compressed opposite portions of elongate member 15 on opposite sides of bend 16 spring outwards until the feet 18, 19 engage opposing wall regions of the nasal cavity 14, as in the previous embodiments, with foot 18 engaging area 23 including the sphenopalatine artery and foot 19 engaging an opposing area of the nasal septum 26. At the same time, extension 46 springs away from member 15, engaging and retracting the middle turbinate 32, as illustrated in FIG. 12. The turbinate extension 46 allows for turbinate medialization during surgery and in the postoperative period if desired. The extension 46 improves intraoperative visualization and stents the middle meatus open in the postoperative period. In an alternative embodiment, turbinate extension 46 may be of a malleable material that is physically bent into the desired position after insertion of the device.

In the above embodiments, the foot or feet may also contain a suitable medication and may slowly release the medication during and after surgery. For example, the tissue engaging surfaces of the feet may have a drug-eluting coating of a material which elutes a drug onto the adjacent tissue, such as a sinus treatment drug, an infection or inflammation combating drug, or a drug which reduces bleeding, or an anesthetic. The medication is designed to be gradually released from the feet onto the adjacent cavity wall surfaces over time.

FIGS. 13A to 13M illustrate some alternative embodiments of the tamponade device which have different configurations for the tissue engaging feet. The tamponade device is otherwise the same as described above in the previous embodiments and is positioned in the same way. In FIG. 13A, the device has round feet 50 which are coplanar with the elongate member or wire and may be formed by bending the wire to form a loop at each end. In FIG. 13B, the feet 52 are square and may be formed by bending the wire to form a square loop at each end. Feet 54 of FIG. 13C are fork-like extensions of the elongate member with two prongs 55 at each end. In FIG. 13D, the feet are V-shaped extensions 56 of the wire or elongate member of the device. The feet 58 in the device of FIG. 13E are round or ovoid solid members. In FIG. 13F, the device has opposing feet 60 of generally rectangular shape with cavities 62. In FIG. 13G, the feet 64 are each formed by a series of spaced cross bars 65 at the ends of the elongate member or wire, and may have a spring-like action. The feet 66 in FIG. 13H are of similar shape to those of FIG. 13B, but each square loop has opposing prongs 68 on each side. The device of FIG. 13I has opposite, triangular loop shaped ends or feet 70 which may be formed by bending the wire into the appropriately shaped loop.

In FIG. 13J, the end feet 72 are cross-shaped with a prong 74 at each end. FIG. 13K shows end feet 75 comprising generally rectangular blocks or pads with the end portions 76 of the elongate member or wire embedded in the respective blocks. FIG. 13L is a hybrid version with two different shaped feet 75, 78, with foot 75 being identical to the feet 75 in FIG. 13K, and foot 78 comprising a bent end portion of the elongate member or wire. FIG. 13M illustrates a modification of the embodiment of FIG. 13L in which the feet 80,81 are both generally rectangular in shape but of different dimensions, with the foot 81 which engages the tissue area including the artery being more elongated than foot 80. Each of the different configurations for tissue-engaging feet illustrated in FIGS. 13A through 13J could be produced by stamping, cutting, selective chemical etching or other suitable means from ribbon or sheet material, thereby eliminating the need for wire-forming or joining operations to produce the entire dual-ended structures illustrated.

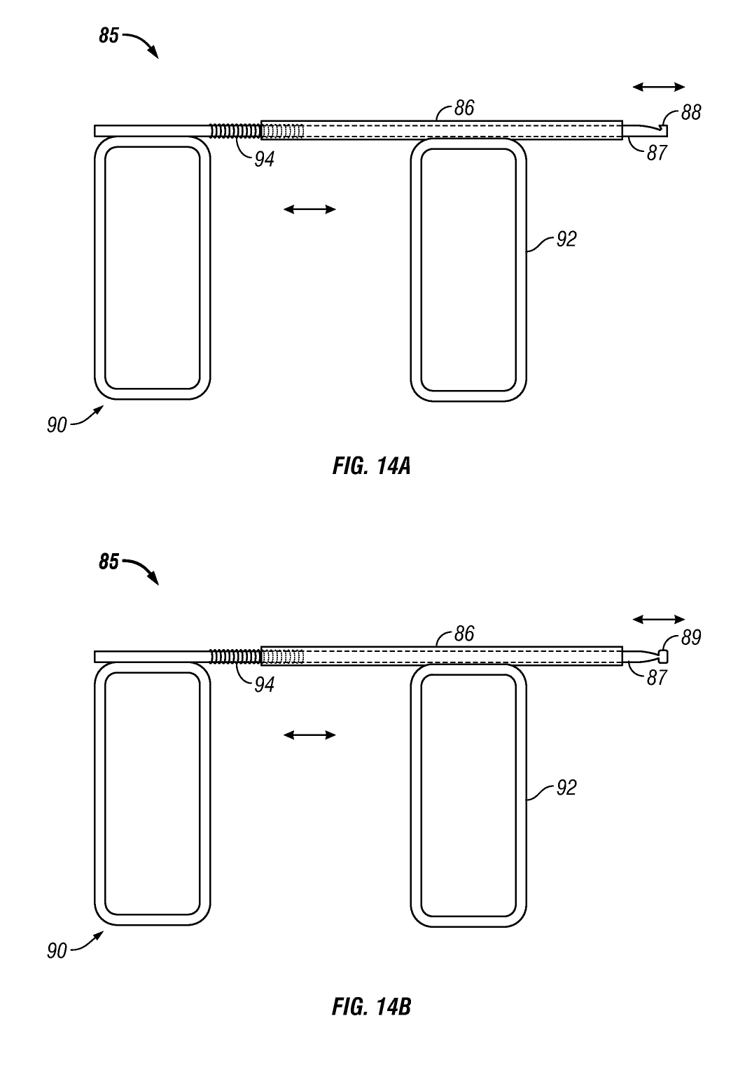

FIG. 14A illustrates another embodiment of an introducer or insertion tool 85 for the arterial tamponade device of any of the above embodiments. Insertion tool 85 is similar to insertion device 25 of FIG. 7A to 7C and comprises a tubular sheath or endoscope 86 in which the device 10 is retained (as in FIG. 7A) as it is inserted into the nasal cavity or other body cavity. In this embodiment, pusher member 87 has a hook 88 at its end which engages over the looped end of the compressed tamponade device when in the position shown in FIG. 7A. FIG. 14B illustrates a modification of the tool of FIG. 14A with an alternative hooked end 89. All other parts are identical to those of FIG. 14A and have been given like reference numerals. A first actuator 90 is coupled to the pusher 87. A second actuator 92 is coupled to the sheath 86. Movement of the first actuator 90 (rotational or longitudinal movement) relative to second actuator 92 causes movement of the pusher relative to the sheath. A spring 94 or other source of compressive force or resistance can be used to provide a selected amount of resistance to moving the first actuator so as to move the end of the pusher 87 with the hook within a certain range of and beyond the end of the sheath. The resistance can decrease the likelihood of accidentally deploying the tamponade.

The hook or retainer 88 reduces the risk of the device springing out of the sheath too quickly and engaging the wrong positions on the nasal cavity walls. Once the device is properly positioned, the pusher member is advanced to disengage the hook 88 from the elongate wire member, then the pusher is rotated to clear the wire before being retracted back into the sheath. An additional embodiment of the introducer sheath and pusher member may be in a square or rectangular shape to maximize collapsed curvature and thus minimize permanent set of the elongate member of the tamponade device.

In each of the above embodiments, the elongate member extending between the feet or ends of the tamponade device is of bendable, resilient metal or plastics material which is biocompatible, arranged so that when compressed and released, the resilient elongate member seeks to return to the original, relaxed and non-compressed state with sufficient tensile strength to counteract mucosal and arterial wall pressure as well as arterial hydrostatic pressure. In other words, the foot 18 which engages the area of the nasal wall including the sphenopalatine artery is pressed against the mucosa and underlying arterial wall with sufficient force to occlude or at least substantially occlude the artery. The device uses the bony boundaries of the nasal wall to provide the counter force necessary for stability and pressure. As noted above, the device may be removed and discarded after use. It may be installed prior to surgery in order to reduce blood in the operative field which can interfere with visualization, and may be left in place for a time period after surgery to reduce post-operative bleeding. The device is of low profile so that it is out of the normal surgical field and does not interfere with the surgeon's view of the surgical site. A safety string or tether (not illustrated) may be attached to the device and extend out of the nose for assistance in locating and removing the device after surgery or after sufficient healing has occurred.

In addition to use during surgery for improved visualization purposes, the above devices may also be used for other purposes. For example, the tamponade device may be left in place after surgery for reduction of post-operative bleeding and also as a post-operative stenting device. The device may also be used as an alternative to lengthy periods of nasal packing, invasive arterial embolization, or permanent vascular ligation procedures for treating chronic nose bleeds or refractory epitaxis.

Although the above embodiments describe use of an arterial tamponade device in the nasal cavity, a similar device of appropriately modified dimensions may be used in other regions of the body to temporarily reduce or eliminate blood flow through an artery if needed during surgery, following surgery or other trauma, or due to a medical condition which causes excessive bleeding. The dimensions of the device may be adjusted as appropriate so that feet 18, 19 bear against opposing regions of a selected body cavity with one foot pressing against a part of the cavity wall including an artery or other blood vessel so as to occlude or at least substantially occlude the vessel and reduce or cut off blood flow.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A tamponade device for applying pressure against a predetermined area of a wall or cavity in the body including a blood vessel, comprising:
an elongate member having opposite first and second ends, the elongate member being movable between a collapsed, compressed condition and a fully expanded, relaxed condition in which the ends are spaced further apart than in the collapsed condition, the ends being biased apart in the collapsed condition;
at least the first end of the elongate member having a foot;
the device being of predetermined shape and dimensions configured for placement in a selected body cavity in a compressed condition and for expansion towards the expanded condition when positioned and released at a predetermined location in the cavity between opposing wall regions of the cavity;
the predetermined spacing between opposite ends of the device in the fully expanded condition being greater than the spacing between predetermined opposing wall regions where the device is to be deployed, whereby the foot applies pressure to one of the wall regions which includes a blood vessel when the device is released at the predetermined location in the cavity.

2. The device of claim 1, wherein the elongate member comprises a length of biocompatible, resilient material.

3. The device of claim 2, wherein the material of the elongate member is nitinol.

4. The device of claim 1, wherein the elongate member is substantially straight in the fully expanded, relaxed condition.

5. The device of claim 1, wherein the device is arcuate in the fully expanded, relaxed condition.

6. The device of claim 1, wherein the device is of predetermined dimensions for placement in a nasal cavity and the foot has an outer surface configured to engage a predetermined area of a lateral nasal wall including the sphenopalatine artery.

7. The device of claim 6, further comprising a turbinate extension member having a first end secured to the elongate member and extending towards said foot, the extension member being movable between an expanded condition extending away from the elongate member and a compressed condition pressed against the elongate member.

8. The device of claim 7, wherein the turbinate extension member is of resilient material and is configured to spring away from the elongate member and bear against the middle turbinate when the device is positioned in a nasal cavity with the foot bearing against a region of the lateral nasal wall including the sphenopalatine artery.

9. The device of claim 8, wherein the extension member is of substantially the same thickness and material as the elongate member.

10. The device of claim 7, wherein the turbinate extension member is of malleable material.

11. The device of claim 1, wherein the second end of the elongate member comprises a second foot, whereby the feet bear against opposing wall regions of the body cavity when the device is positioned at a predetermined location in the cavity.

12. The device of claim 1, wherein the foot has an outer surface configured for face to face engagement with a predetermined wall region of a body cavity when the device is deployed in the cavity.

13. The device of claim 12, wherein the outer surface has a roughened, slip resistant surface texture.

14. The device of claim 12, wherein the outer surface has a plurality of dimples.

15. The device of claim 12, wherein the outer surface has a plurality of protrusions.

16. The device of claim 11, wherein the feet have outer surfaces configured for engagement with opposing wall regions of a body cavity when the device is deployed in the cavity.

17. The device of claim 16, wherein the outer surface of each foot has a roughened, slip resistant surface texture.

18. The device of claim 11, wherein the feet are of different sizes.

19. The device of claim 11, wherein the feet are of different shapes.

20. A method of temporarily applying pressure to an area of a body cavity wall including a blood vessel and occluding or substantially occluding the vessel to stop or reduce blood flow, comprising:
holding an expandable tamponade device in an insertion device in a compressed, unexpanded condition;
inserting the compressed tamponade device into a body cavity with a pressure pad at one end of the device oriented to face towards a predetermined region of the cavity wall which includes a blood vessel; and
releasing the compressed tamponade device from the insertion device at a predetermined location in the body cavity whereby the device expands towards a fully expanded condition and opposite ends of the device engage and press against opposing wall regions of the body cavity before the device is fully expanded, whereby the pressure pad presses against the predetermined region of the cavity wall including the blood vessel and applies sufficient pressure to at least partially occlude the blood vessel and reduce blood flow to the body cavity.

21. The method of claim 20, wherein the body cavity is a nasal cavity and the pressure pad is pressed against a predetermined region of the lateral nasal wall that includes the sphenopalatine artery.

22. The method of claim 21, further comprising positioning a turbinate extension of the tamponade device to engage and retract the middle turbinate away from the lateral nasal wall when the device expands towards the fully expanded condition.

23. The method of claim 20, wherein the step of releasing the compressed device comprises allowing the device to expand until pressure pads at opposite ends of the device bear against opposing wall regions of the body cavity to hold the device in place.

24. The method of claim 20, further comprising performing a surgical procedure at the body cavity while the tamponade device is in place, leaving the tamponade device in place for a predetermined time period after surgery, and removing the tamponade device from the body cavity.

25. The method of claim 20, wherein the device is held in a compressed condition in an insertion sheath while the device and sheath are inserted into the body cavity, and the step of releasing the device comprises pushing the device out of the sheath into the predetermined location in the body cavity.

26. The method of claim 25, wherein the device comprises a resilient elongate member and the step of holding the device in a compressed, unexpanded condition comprises bending the elongate member from a substantially straight, relaxed and expanded condition into a bent condition in which opposite ends of the device are moved towards one another.

27. The method of claim 26, wherein the step of pushing the tamponade device out of the sheath is carried out by a reciprocating pusher in the sheath which is movable between a retracted position in the sheath and an extended position projecting out of the sheath.

28. The method of claim 27, further comprising holding a bent portion of the elongate member with a hook at the end of the pusher until the tamponade device completely exits the sheath, and releasing the elongate member from the hook and retracting the pusher into the sheath when the tamponade device is in position.

29. An intranasal tamponade device for applying pressure against a predetermined area of a nasal cavity including the sphenopalatine artery, comprising:
an elongate, expandable member having opposite first and second ends, the elongate member being movable between a collapsed, compressed condition and a fully expanded, relaxed condition in which the ends are spaced further apart than in the collapsed condition, the ends being biased apart in the collapsed condition;
at least the first end of the elongate member having a foot;
the device being of predetermined shape and dimensions configured for placement in a nasal cavity in a compressed condition and for expansion towards the expanded condition when positioned and released at a predetermined location in the cavity between opposing wall regions of the cavity; and
the predetermined spacing between opposite ends of the device in the fully expanded condition being greater than the spacing between predetermined opposing wall regions of the nasal cavity where the device is to be deployed, whereby the foot applies pressure to one of the wall regions which includes the sphenopalatine artery when the device is released at the predetermined location in the cavity.

30. The device of claim 29, wherein the spacing between opposite ends of the device in the fully expanded condition is at least 20 mm.

31. The device of claim 30, wherein the spacing between opposite ends of the device in the fully expanded condition is approximately 25 mm.

32. The device of claim 29, wherein the foot comprises an enlarged end pad secured to the elongate member, the pad having a tissue engaging surface.

33. The device of claim 32, wherein the tissue engaging surface is circular and has a diameter in the range from 5 to 12 mm.

34. The device of claim 33, wherein the diameter is 7.5 mm.

35. The device of claim 32, wherein the tissue engaging surface is oval and has an elongate axis of length in the range from 5 to 12 mm.

36. The device of claim 32, wherein the tissue engaging surface is rectangular and has a length in the range from 5 to 12 mm.

37. The device of claim 29, wherein the foot is an extension of the elongate member.

38. The device of claim 37, wherein the elongate member is a wire and the foot comprises a bent end portion of the wire.

39. The device of claim 38, wherein the foot is fork-shaped.

40. The device of claim 38, wherein the foot is Y-shaped.

41. The device of claim 29, wherein the foot is T-shaped.

42. The device of claim 29, wherein the second end of the elongate member comprises a second foot, whereby the feet bear against opposing wall regions of the body cavity when the device is positioned at a predetermined location in the cavity.

43. The device of claim 42, wherein the feet are of the same shape and dimensions.

44. The device of claim 42, wherein the feet are of different sizes.

45. The device of claim 42, wherein the first foot comprises a bent end portion of the elongate member and the second foot comprises a tissue engaging pad secured the second end of the elongate member.

46. The device of claim 42, wherein both feet comprise rectangular pads and the first foot has a length greater than the second foot.
